(19) United States
(12) Patent Application Publication
(10) Pub. No.: US 2015/0139938 A1
(43) Pub. Date: May 21, 2015

(54) Methods and Compositions for Improving Pial Collateral Circulation and Treating Blood Clotting Disorders

(71) Applicant: Ohio State Innovation Foundation, Columbus, OH (US)

(72) Inventors: Chandan Sen, New Albany, OH (US); Cameron Rink, Galloway, OH (US); Sashwati Roy, New Albany, OH (US); Greg Christoforidis, Columbus, OH (US)

(73) Assignee: Ohio State Innovation Foundation, Columbus, OH (US)

(21) Appl. No.: 14/406,402

(22) PCT Filed: Jun. 7, 2013
(86) PCT No.: PCT/US13/44690

§ 371 Date: Dec. 8, 2014

Related U.S. Application Data
(60) Provisional application No. 61/657,433, filed on Jun. 8, 2012.

(51) Int. Cl. A61K 31/355; A23L 1/29; A61K 45/06

(52) U.S. Cl. CPC A61K 31/355; A61K 45/06; A23L 1/296; A23V 2002/00
USPC 424/85.1; 514/458; 514/56; 424/766; 424/729; 424/754; 424/643; 424/682; 424/655; 424/646

(57) ABSTRACT

The present invention provides methods of promoting arteriogenesis in a subject. Embodiments include methods comprising: administering an effective dose of tocotrienol to the subject; causing an increase in Tissue Inhibitor of Metalloproteinase Metallopeptidase Inhibitor 1 (TIMP1) in vessels of cerebrovascular collateral circulation in the subject; attenuating the activity of Matrix Metalloproteinase-2 (MMP2); thereby promoting arteriogenesis.

### CROSS REFERENCE TO RELATED APPLICATIONS

This application claims the benefit of U.S. Provisional Patent Application No. 61/657,433, filed Jun. 8, 2012, the entire disclosure of which is expressly incorporated herein by reference for all purposes.

### STATEMENT REGARDING FEDERALLY SPONSORED RESEARCH

This invention was made with U.S. government support under grant UL1RR025755 and NIH grant NS42617. The government has certain rights in this invention.

### REFERENCE TO SEQUENCE LISTING

This application is being filed electronically via the USPTO EFS-WEB server, as authorized and set forth in MPEP§1730 II.B.2(a)(A), and this electronic filing includes an electronically submitted sequence (SEQ ID) listing. The entire content of this sequence listing is herein incorporated by reference for all purposes. The sequence listing is identified on the electronically filed .txt file as follows: 604_{—}53835_SEQ_ID_OSU-2009-085.txt, created on May 29, 2013 and is 3,492 bytes in size.

### BACKGROUND OF THE INVENTION

Of the 795,000 cases of stroke each year in the United States, ˜25% are repeat stroke events. In addition, fifteen percent of all stroke events are preceded by a transient ischemic attack (TIA), defined as a temporary episode of neurologic dysfunction caused by reduced blood flow to the brain, but without permanent damage to brain tissue. After a TIA, the 90-day risk of stroke is as high as 17.3%. Thus, prophylactic interventions may play a key role in favorably modifying stroke outcomes, especially for those who have already suffered from a TIA and, therefore, are facing a high risk of a major stroke event.

Clinical trials testing the effects of vitamin E in a wide range of major health disorders have come to the general conclusion that vitamin E either is not helpful or could be harmful under certain conditions. Meta-analyses of over 20 randomized, controlled clinical trials testing vitamin E have now reached conclusions that, on one hand, serve the basis for readjusting public policies and practices, while on the other, suffer from a major blind spot which is not recognized in any of these reports. While title claims of such meta-analyses address vitamin E as whole, they fail to recognize that the only form of vitamin E studied in all these trials is α-tocopherol which represents one-eighth of the natural vitamin E family. Natural vitamin E exists in two forms: tocopherols and tocotrienols. Both tocopherols and tocotrienols possess a chromanol ring. Within the tocopherol and tocotrienol families, the isoforms are differentiated as α, β, γ, and δ according to the presence of methyl groups at positions 5, 7, and 8, respectively. Tocopherols are characterized by a saturated side chain, whereas tocotrienols possess an isoprenoid side chain with double bonds at C-3, -7 and -11.

Recent interest in the biological properties of tocotrienol has sharply risen because of the unique biological functions of this form of natural vitamin E not shared by the better known tocopherols which have failed to live up to expectations in clinical trials. At nanomolar concentrations, α-tocotrienol (αTCT) but not α-tocopherol, is a potent neuroprotective agent. On a concentration basis, this represents the most potent of all biological functions of the entire vitamin E family. Neural cell biology studies have identified unique αTCT-sensitive signaling checkpoints that rescue cells from inducible cell death caused by a range of insults. Importantly, although αTCP is detected in serum from subjects who receive it from dietary sources, the presence of αTCT in the serum of non-supplemented Americans is negligible. This is likely due to Western food consumption, as most Western food contains very low levels of tocotrienol. As the biological importance of TCT is increasingly shown, there is a need for evidence-based formulations and methods of administering TCT to optimize health and aid in disease management.

Molecular mechanisms of postnatal collateral growth and remodeling, termed arteriogenesis, are distinct from those invoked in angiogenesis and vasculogenesis. Angiogenesis describes the formation of new capillaries, and vasculogenesis is the embryonic development of blood vessels from angioblasts. Arteriogenesis describes the formation of mature arteries from existing arterioles after an arterial occlusion. It shares some features with angiogenesis, but the pathways leading to it are different, as are the final results: arteriogenesis is potentially able to fully replace an occluded artery whereas angiogenesis cannot. Under special circumstances, arteriogenesis may lead to the recovery of blood flow from markedly reduced levels. Increasing the number of capillaries within an ischemic region cannot increase blood flow when an occlusion is upstream. Another fundamental difference between the two types of vascular growth is angiogenesis' dependency on tissue hypoxia/ischemia. In contrast, arteriogenesis occurs in an oxygenated environment. There is a need for further elucidation of factors influencing angiogenesis and methods of therapeutic intervention to treat and prevent ischemic stroke.

### SUMMARY OF THE INVENTION

The present invention provides methods of improving pial collateral circulation and protecting ischemic tissue, comprising: a) administering a composition comprising at least one form of tocotrienol in an amount of from about 10 mg to about 1000 mg per day to a subject in need of pial collateral circulation improvement and ischemic tissue protection; and b.) improving pial collateral circulation and protecting ischemic tissue in the subject.

The present invention also provides methods of promoting arteriogenesis in a subject comprising: administering a composition comprising at least one form of tocotrienol in an amount of from about 10 mg to about 1000 mg per day to a subject in need of arteriogenesis promotion; and promoting arteriogenesis in the subject.

The present invention also provides methods of increasing Tissue Inhibitor of Metalloproteinase Metallopeptidase Inhibitor 1 (TIMP1) in vessels of cerebrovascular collateral circulation and attenuating the activity of Matrix Metalloproteinase-2 (MMP2) in a subject in need thereof, comprising: administering a composition comprising at least one form of tocotrienol in an amount of from about 10 mg to about 1000 mg per day to a subject in need of an increase in Tissue Inhibitor of Metalloproteinase Metallopeptidase Inhibitor 1 (TIMP1) in vessels of cerebrovascular collateral circulation and in need of attenuation of the activity of Matrix Metalloproteinase-2 (MMP2); and increasing Tissue Inhibitor of Metalloproteinase Metallopeptidase Inhibitor 1 (TIMP1) in vessels of cerebrovascular collateral circulation and attenuating the activity of Matrix Metalloproteinase-2 (MMP2) in the subject.

The present invention also provides methods of ameliorating the symptoms of cerebral blood clotting, or reducing the risk of cerebral blood clotting, in a subject comprising: administering a composition comprising at least one form of tocotrienol in an amount of from about 10 mg to about 1000 mg per day to a subject in need of amelioration of the symptoms of cerebral blood clotting, or reducing of risk of cerebral blood clotting; and ameliorating the symptoms of cerebral clotting, or reducing risk of cerebral blood clotting in the subject.

Also provided are such methods, wherein the cerebral blood clotting is associated with a cerebrovascular ischemic disease, wherein the disease is selected from the group consisting of: (a) cerebral ischemia, in particular transient ischemic attack, stroke, vascular dementia and/or infarct dementia; (b) myocardial ischemia, in particular a coronary heart disease and/or myocardial infarction; and/or (c) peripheral limb disease, in particular periphery arterial occlusive disease.

Also provided are such methods, which further have an effect on the subject selected from the group consisting of: attenuating ischemic stroke-induced lesion volume; preventing loss of white matter fiber tract connectivity following stroke; improving cerebrovascular collateral circulation; preventing blood vessel injury; reducing the risk of ischemic stroke; reducing cerebrovascular ischemic disease; and ameliorating the symptoms of obstruction of a blood vessel.

Also provided are such methods, wherein 100 mg and 500 mg per day of at least one tocotrienol is administered via oral supplementation, for at least four weeks.

Also provided are such methods, wherein the at least one tocotrienol is selected from the group consisting of: α tocotrienol; β tocotrienol; γ tocotrienol; δ tocotrienol; and combinations thereof.

Also provided are such methods, wherein the composition comprises mixed tocotrienols enriched to a percentage of the total weight of the composition selected from the group consisting of: approximately 20%; approximately 30%; approximately 40%; approximately 50%; approximately 60%; approximately 70%; approximately 80%; and approximately 90%.

Also provided are such methods, wherein the at least one tocotrienol is administered as 400 mg daily dose of Tocovid Suprabio®, for at least four weeks.

Also provided are such methods, wherein the oral supplement is delivered by one or more of: a capsule; a tablet pill; a colloid; a piece of chewing gum; a gel; a drink; a food additive; a thin film dissolving strip; an emulsified food spread; an emulsion, a syrup; a meat food; a dairy food; and an egg.

Also provided are such methods, wherein the subject is at elevated risk for cerebral blood clotting.

Also provided are such methods, which further comprises administering a blood-thinning agent.

Also provided are such methods, wherein the subject is selected from the group consisting of: human; livestock; companion animal; research animal.

Also provided are such methods, wherein the subject is selected from the group consisting of: astronaut; pilot; professional racecar driver; deep-sea diver; mountain climber; pre-surgery patient; sickle-cell anemia patient; sleep apnea patient; drug rehabilitation patient; elderly person; elderly animal; greyhound; or racehorse.

Also provided are such methods, wherein said subject has an attribute selected from the group consisting of: (a) showing symptoms of being at risk of developing the cerebrovascular ischemic disease; (b) showing any risk markers in ex vivo tests, in particular in blood samples; (c) has previously had a cerebral or myocardial ischemia; and/or (d) has a predisposition of developing a cerebrovascular ischemic disease, in particular a genetic predisposition.

Also provided are such methods, wherein the symptoms are selected from the group comprising: neurological malfunctions, transitory ischemic attack, congestive heart failure, angina pectoris, valvular heart disease, cardiomyopathy, pericardial disease, congenital heart disease, coarctation, atrial and/or ventricular septal defects.

Also provided are such methods, wherein the subject exhibits at least one condition selected from the group consisting of Alzheimer's disease; sclerosis, in particular atherosclerosis and/or transplantation-induced sclerosis; a cerebral occlusive disease, renal occlusive disease, a mesenterial artery insufficiency or an ophthalmic or retinal occlusion, post-operative or post-traumatic condition; thrombosis; embolism; restenosis, in particular primary restenosis, secondary restenosis and/or in-stent restenosis; trisomy 21; hypoglycemia; vasculitis; preeclampsia; placental hypoxia; sleep apnea; sexual dysfunction, in particular erectile dysfunction or female sexual dysfunction; post-operative hypoxia; Raynaud's disease; endothelial dysfunction; cancer; renal failure; varicose veins; edema; hypotension; decubitus; carbon monoxide poisoning; heavy metal poisoning; ulcers; sudden infant death syndrome; erythroblastosis; asthma; chronic obstructive pulmonary disease; sickle cell disease; induced g-forces which restrict the blood flow and force the blood to the extremities of the body; caisson's disease; localized extreme cold, in particular frost bite; tourniquet application; an increased level of glutamate receptor stimulation or for any disease where atherosclerosic plaques in the vascular wall lead to an obstruction of the vessel diameter; osteonecrosis; and Legg Calvé Perthes disease.

Also provided are such methods, wherein said subject has been, or will be, exposed to: (a) a pharmaceutical or medical treatment damaging one or more arteries; (b) a radiation treatment damaging one or more arteries; and/or (c) a surgical treatment damaging one or more arteries.

The present invention also provides pharmaceutical compositions for the treatment of an obstruction in a blood vessel comprising: one or more thrombolytic drugs, and one or more tocotrienol-comprising composition.

Also provided are such compositions, wherein the tocotrienol-comprising composition is Tocovid Suprabio®.

Also provided are such compositions, which further comprise one or more compounds selected from the group consisting of: heparin; tocopherols; one or more colony stimulating factor(s) (CSF(s)); one or more icosanoid(s); angiotensin converting enzyme (ACE) inhibitors; beta-blockers; antiplatelet agents; pentoxifylline; and cilostazol.

Also provided are nutritional supplements to aid circulatory health comprising, at least one tocotrienol and at least two additional compounds selected from: vitamin A, vitamin B, vitamin C, vitamin D, grape seed extract, hawthorn extract, green tea extract, garlic extract, limonene, carnitine, lutein, zeaxanthin, omega-3 essential fatty acids, zinc, calcium, chromium, and iron.

Also provided are such supplements, wherein the additional compounds selected are fat-soluble.

Also provided are infant formula compositions comprising fat, carbohydrate, protein, and vitamins wherein at least one of the vitamins is a tocotrienol.

### BRIEF DESCRIPTION OF THE DRAWINGS

The patent or application file may contain one or more drawings executed in color and/or one or more photographs. Copies of this patent or patent application publication with color drawing(s) and/or photograph(s) will be provided by the U.S. Patent Office upon request and payment of the necessary fee.

FIG. 1A-FIG. 1E. Tocotrienol enriched natural vitamin E protects against stroke-induced brain injury. (A and B) Effect of 10 week oral supplementation on cerebral cortex concentration of tocotrienols and tocopherols. (A) No tocotrienols were detected in brain of placebo (PBO) supplemented canines. Tocotrienol enriched (TE) supplementation significantly increased α-, γ-, and δ-tocotrienol isomers in cerebral cortex. (B) A moderate, but significant (*p=0.047) increase in brain α-tocopherol level was observed as each TE gel capsule contains 61.5 mg of α-tocopherol. (C) Stroke-induced infarct volume in response to stroke. MD=mean diffusivity map taken at 1 h, FLAIR=fluid attenuated inversion recovery taken at 24 h. Representative coronal slice MR images of canine brain at (D) 1 h demonstrating cytotoxic edema (*p<0.05) and (E) 24 h demonstrating cytotoxic and vasogenic edema following reperfusion (*p<0.005).

FIG. 2A-FIG. 2C. TE attenuates white matter injury following acute ischemic stroke. (A) Streamline tractography of PBO and TE white matter fiber tracts was performed with 2 ROI masks to visualize tracts connecting the corona radiata to the internal capsule at 24 h. Fiber tracts were overlaid on T2-weighted structural scan (512×512 matrix) to visualize in context of contralateral (right) and ipsilateral (left) hemispheres in the coronal orientation. Sagittal views of contralateral and ipsilateral hemispheres demonstrate the protective effect of TE supplementation. (B) Probabilistic tractography reveals connectivity of white matter fiber tracts projecting from the seed region of the internal capsule to the corona radiata in representative canines. Color shift, from black to red to yellow to white, denotes a higher degree of relative connectivity between regions in the stroke affected hemisphere of PBO and TE supplemented canines. (C) Variance of probabilistic tracts as a function of the distance from the internal capsule seed region. contra=contralateral, ipsi=ipsilateral.

FIG. 3A-FIG. 3L. TE improves cerebrovascular collateral circulation during acute ischemic stroke. Cerebrovascular collaterals were identified by digital subtraction angiography (DSA) in PBO (A-E) and TE (F-J) treated canines. To visualize collaterals of the stroke-affected MCA territory (green lines), pre-stroke arterial (A, F) and venous (B, G) DSA of left internal carotid artery (L-ICA) were compared to post-stroke arterial (D, I) and venous (E, J) DSA of right internal carotid artery (R-ICA). Post-stroke L-ICA DSA during the arterial phase (C, H) demonstrates effective MCA occlusion by embolic coil (marked by red oval). During the post-stroke arterial phase, greater collateral perfusion (black arrow) was observed in MCA territory of TE supplemented canines as compared to PBO controls (I vs. D). Likewise, more venous flow and contrast “blush” (black triangle) was observed in stroke-affected hemisphere of TE supplemented canines (J vs E). Mean collateral score for PBO and TE supplemented canines was determined according to an 11-point scale (methods). (K) Collateral score during stroke was significantly higher in TE supplemented canines as compared to PBO controls. *p<0.05. (L) Collateral score correlation with infarct volume (coefficient of determination, r2=0.821), open diamonds represent PBO, closed diamonds represent TE canines.

FIG. 4A-FIG. 41I. TE increases expression of arteriogenic markers in laser capture isolated cortex arterioles. (A-C) Arterioles (arrows, mean diameter 6.6±2.9 μm) were selectively captured from contralateral control and ipsilateral stroke-affected cerebral cortex 24 h after stroke onset. (D) To verify specificity of captured elements, gene expression of vessel marker (VWF), neuron marker (NF-H), and glial marker (GFAP) was checked with real-time PCR. *p<0.05 VWF vs NF-H, ND=not detected. (E-H) Expression of arteriogenic genes was validated using real-time PCR in contralateral (white) and ipsilateral (black) arterioles. *p<0.05 in TE supplemented control vs. stroke. †p<0.05 in PBO vs. TE control tissue. (E) Chloride intracellular channel 1 (CLIC1). (F) Chloride intracellular channel 4 (CLIC4). (G) Tissue inhibitor of metalloproteinase 1 (TIMP1). (H) Vascular endothelial growth factor (VEGF).

FIG. 5A-FIG. 5D. TE inhibits MMP2 activity in stroke-affected cerebral cortex. No difference in MMP2 protein expression was observed by Western blot (A) and densitometric analysis (B) of contralateral (contra) and ipsilateral (ipsi) somatosensory cortex of PBO and TE supplemented canines 24 h after stroke. Gelatin zymography (C) and densitometry (D) demonstrates significantly higher MMP-2 activity in stroke-affected hemisphere of PBO, not TE canines. *p<0.05 PBO cont vs. stroke, †p<0.05 PBO stroke vs. TE stroke.

FIG. 6. Physiological parameters. Canine physiological parameters were assessed at baseline (prior to emolic coil occlusion of the MCA), during ischemia, and immediately following reperfusion.

FIG. 7. Primer sequences for real-time PCR. (SEQ ID NOs:1-14, respectively in order of appearance.)

FIG. 8A-FIG. 8F. Fluoroscopic guidance of middle cerebal artery occlusion in canine. Under guided C-arm fluoroscopy, (A) the microwire is advanced from the basilar artery (BA), along the posterior communicating artery (PCOM) to the left middle cerebral artery (L-MCA). (B) Following BA contrast injection, DSA permits visualization of the Circle of Willis, overlaying the path of the microwire into the L-MCA. A microcatheter is tracked along the microwire into the L-MCA. From the microcatheter the embolic coil (C) is deployed into the L-MCA, occluding the M1 segment. (D) DSA of L-ICA contrast injection confirms that the L-MCA is occluded. (E) DSA of R-ICA contrast injection confirms that the contralateral MCA is still patent. Following 1 hour of occlusion, the embolic coil is retrieved to reperfuse the stroke-affected hemisphere. (F) BA Contrast injection reveals that the Circle of Willis is intact. The L-MCA perfuses notable slower than the RMCA; emblematic of the onset of edema and successful focal acute ischemic stroke.

FIG. 9. 3D volumetric reconstruction of FLAIR images from representative PBO and TE canine 24 hours after stroke.

FIG. 10. 3D volumetric reconstruction of streamline tractography from representative PBO and TE canine 24 hours after stroke.

### DETAILED DESCRIPTION OF THE INVENTION

Embodiments of the present invention provide a prophylactic intervention to improve collateral circulation during acute ischemic stroke.

Vitamin E consists of tocopherols and tocotrienols where α-tocotrienol is the most potent neuroprotective form that is also effective in protecting against stroke in rodents. As neuroprotective agents alone are insufficient to protect against stroke, the inventors tested the effects of tocotrienol on the cerebrovascular circulation during ischemic stroke using a pre-clinical model that enables fluoroscopy-guided angiography.

Mongrel canines (mean weight=26.3±3.2 kg) were supplemented with tocotrienol enriched (TE) supplement (200 mg b.i.d, n=11) or vehicle placebo (PBO, n=9) for ten weeks prior to inducing transient middle cerebral artery (MCA) occlusion. MRI was performed 1 h and 24 h post-reperfusion to assess stroke-induced lesion volume. TE supplementation significantly attenuated ischemic stroke-induced lesion volume (p<0.005). Furthermore, TE prevented loss of white matter fiber tract connectivity following stroke as evident by probabilistic tractography. Post-hoc analysis of cerebral angiograms during MCA occlusion revealed that TE supplemented canines had improved cerebrovascular collateral circulation to the ischemic MCA territory (p<0.05). TE induced arteriogenic TIMP1 and subsequently attenuated the activity of MMP2. Arteriogenic effects of TE are beneficial to patients who have suffered from transient ischemic attack and are therefore at a high risk for stroke.

Emblematic of the high morbidity and mortality associated with stroke are the failures of potential stroke therapeutics which showed benefit in small animal rodent stroke models but failed to translate into clinical success. As a result, rodent stroke models have been criticized for the anatomical disparity between small and large mammalian brains, large variability in infarct volumes, and inaccurate methods of inducing and confirming arterial occlusion. As compared to the lissencephalic brain of rodents, the size and anatomical feature set of the canine brain closely mimics that of humans. Canines have a highly evolved gyrencephalic neocortex with a white to gray matter ratio that closely approximates primates, and like humans, collateral circulation in the middle cerebral artery (MCA) territory has been documented in canines. Furthermore, the current experimental model benefits from C-arm fluoroscopy visualization of middle cerebral artery occlusion (MCAO). As opposed to the widely used rodent intraluminal thread model of MCAO, this method permits repeated real-time documentation of the stroke event, improving the overall reproducibility of the procedure and enabling objective assessment of collateral circulation during cerebral ischemia. The latter proved to be pivotal in identifying the effects of TE on perfusion of the stroke-affected brain tissue. Until this point, the current literature documents protective effects of stroke in vivo on the basis of TE's neuroprotective properties. αTCT specific mechanisms of neuroprotection depend on three key cytosolic targets involved in glutamate excitotoxicity and neurodegeneration: c-Src kinase (c-Src), 12-lipoxygenase (12-Lox), and phospholipase A2 (PLA2). Neuroprotectants alone, however, are thought to be insufficient in providing meaningful protection against stroke. Multi-modal therapies that target both neuro and vascular pathophysiology are desirable.

The cerebrovascular collateral circulation refers to a subsidiary network of small vascular channels that can stabilize cerebral blood flow when principal conduits are obstructed, as in ischemic stroke. These small collateral pathways can occur through leptomeningeal arterioles that overlap and anastomose distal branches of the anterior and posterior cerebral arteries (ACA, PCA) with the MCA. Indeed, the risk and severity of stroke-mediated pathology is worse in patients with poor collateral circulation. The mechanistic process in which pre-existing arterioles are recruited to bypass the site of occlusion is termed arteriogenesis. Arteriogenesis invokes a rapid proliferative and remodeling response that is distinct from passive dilatation, developmental vasculogenesis, or neovascular angiogenesis. Induction of arteriogenic collateral growth in the brain occurs as early as 24 h following vessel occlusion and the onset of adaptive arteriogenesis is marked by early-phase expression of protease inhibitor TIMP1 in growing collaterals of the brain. TE supplementation significantly increased TIMP1 expression in both contralateral control and stroke-affected arterioles of the cerebral cortex.

Provided herein is a regimen of TE supplementation that regulates TIMP1 expression and subsequently invokes cerebrovascular arteriogenesis.

Diffusion tensor imaging (DTI) enables repeated, non-invasive assessment of white matter cytoarchitecture and connectivity due to unrestricted parallel (anisotropic) diffusion of water molecules along axonal fiber tracts. This magnetic resonance imaging MRI-based technique has emerged as a clinically relevant tool for the prognostic diagnosis of neurological deficit and assessment of rehabilitation potential in stroke patients. Proceeding from the cortex, white matter fiber tracts of the corona radiata, or “radiating crown”, converge and pass between the lenticular nucleus and thalamus in the form of a band called the internal capsule. The fiber tracts of the corona radiata and internal capsule contain corticospinal nerve bundles that are responsible for sensorimotor neurotransmission between somatosensory cortex and motor neurons.

Streamlined and probabilistic tractography were employed to assess stroke-mediated injury and loss of white matter connectivity between internal capsule and the corona radiata following stroke. White matter of TE treated animals, not PBO, maintained the cytoarchitectural connection between internal capsule and corona radiata, suggesting that TE protected anatomical connectivity, and therefore biological function, from stroke injury. Taken together with the marked improvement in functional outcomes following MCAO in TE supplemented mice, data show that prophylactic TE supplementation attenuates the severity of stroke-associated sensorimotor injury.

In addition to tractography, DTI also enables assessment of stroke-induced lesion. During the acute phase of cerebral ischemia (0-24 h post-reperfusion), a decline in apparent diffusion coefficient (ADC) maps generated from DTI is associated with cytotoxic edema causing irreversible brain injury. Using DTI imaging immediately following stroke reperfusion, the inventors found that TE supplementation attenuated stroke-induced cytotoxic edema within the first hour following reperfusion. While cytotoxic edema evolves over minutes to hours, vasogenic edema occurs over hours to days and is associated with blood brain barrier disruption. MRI performed at 24 h employed a T2-weighted fluid attenuated inversion recovery (FLAIR) sequence that captures both cytotoxic and vasogenic components of stroke-induced edema. Lesion volume in TE supplemented animals did not significantly increase between 1 h and 24 h MRI; the TE largely prevented blood brain barrier disruption and subsequent vasogenic edema.

As a nutrient, tocotrienols have been safely consumed by humans, especially in the Far East, for many years. Furthermore, tocotrienols have been Generally Recognized As Safe (GRAS, GRN No. 307) certified by the United States FDA as ingredients in food. In nature, tocopherols and tocotrienols are found in abundance throughout the plant kingdom. Tocopherols are the primary source of vitamin E in photosynthetic plant tissue, while tocotrienols are enriched in endosperm of cereals, grains, and palm seed. A growing body of studies support that different members of the natural vitamin E family may have unique biological properties relevant to health and disease. For example, anti-tumorigenic properties of γ-tocotrienol, not shared by α-tocopherol, have been described in both breast and prostate cancer. Furthermore, tocotrienol transport to tissue, including brain, has been reported in the absence of tocopherol transfer protein (TTP), the transport system with high affinity for α-tocopherol. Indeed, loss of fertility in TTP−/− mice could be rescued by TE supplementation. At a time when meta-analyses of clinical trials testing the effect of tocopherols in a variety of disease setting draw major conclusions relevant to public health policies and practices, this illuminates a blind spot in research: that generalized claims on vitamin E should instead be limited to the specific form of vitamin E studied.

Demonstrated herein is that prophylactic supplementation of natural vitamin E tocotrienols reduces brain injury following stroke in a pre-clinical setting. Given the observed effect of TE in improving collateral circulation during cerebral ischemia and the established hypo-cholesterolemic effects of tocotrienol supplementation, protocols to induce the effects of prophylactic TE supplementation on reducing stroke incidence are provided herein. Therefore, beneficial effects are shown for supplementation of TE in a high-risk stroke population, such as TIA patients. With more than 200,000 Americans each year, the TIA patient population is well suited for treatment.

### Examples

Statin-mimetic cholesterol lowering properties of αTCT in humans, in addition to neuroprotection, positions tocotrienol as a strong candidate for stroke therapeutics. The inventors have demonstrated that orally supplemented αTCT protects against stroke-induced lesion in the brain of spontaneously hypertensive rats. As small animal studies are recognized to be of limited reliability to predict success for stroke therapeutics in clinical trials, the inventors developed a minimally invasive pre-clinical canine model to test the efficacy of a tocotrienol enriched supplement (TE) in a randomized, blind, placebo controlled setting. Angiography, enabled in the inventors' large animal setting, helped elucidate that prophylactic TE supplementation improves collateral blood flow to the stroke-affected territory during stroke. In the clinic, angiographic collateral grading has been used as a predictor of stroke outcome. Molecular mechanisms of postnatal collateral growth and remodeling, termed arteriogenesis, are distinct from those invoked in angiogenesis and vasculogenesis. Outcomes of the current research demonstrate a direct link between tocotrienol supplementation and the expression of pro-arteriogenic factors in perfused collaterals of the stroke-affected hemisphere.

### Example 1

### Randomized, Blind, Placebo Controlled, Supplementation Regimen

All experimentation was approved by the Institutional Animal Care and Use Committee of The Ohio State University. Twenty mongrel canines (2.4±0.9 yrs, 26.6±2.6 kg) were subjected to gross physical, heartworm, complete blood count, and blood chemistry tests by veterinary faculty of The Ohio State University prior to study inclusion. No gross physical abnormalities, heartworm, or significant differences in complete blood count or blood chemistry were observed by veterinary staff. Following baseline physicals, canines were randomized into two treatment groups—one receiving TE (n=11, 200 mg mixed tocotrienols, Carotech Inc, Malaysia), and the other receiving vitamin E deficient corn oil (n=9, vehicle placebo, PBO). Canines were maintained on standard chow (TD2025; Harlan Teklad) for the duration of the supplementation. TE and PBO supplements were delivered orally in gel capsules that were identical in appearance and size. Canines received supplements twice per day, after morning and evening meals, for a period of ten weeks. Stroke was induced within 12 hours after the last supplement was received. Research and veterinary staff were blinded to capsule contents and treatment groups until all MRI stroke outcome data was independently reviewed by faculty of the Center for Biostatistics at The Ohio State University Medical Center.

### Example 2

### C-Arm Fluoroscopy Guided Pre-Clinical Model of Acute Ischemic Stroke

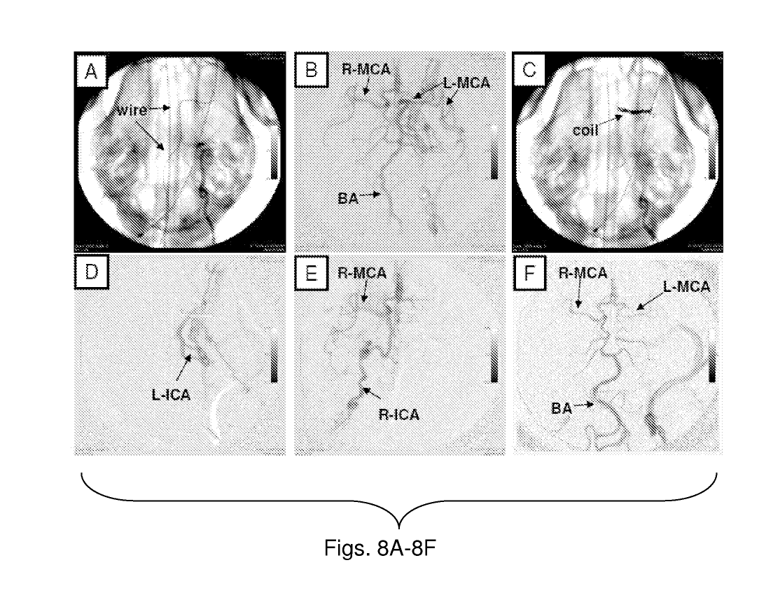

The minimally invasive, endovascular approach to achieve middle cerebral artery occlusion in canines was performed. Briefly, the anesthetized canine (1.5-2.0% isoflurane) underwent bilateral femoral artery access with 5 French sheaths (ArrowGE Healthsystems) from which 4-Fr and 5-Fr guide catheters (Boston Scientific) were used to provide access to the basilar artery (BA) system and for routine contrast (Omnipaque) visualization of the MCA territories. Microcatheter techniques were used to access and occlude the MCA from the BA. An embolic coil (3 mm×20 cm Ultrasoft Matrix2 Platinum Coil, Boston Scientific) was delivered into the M1 segment of either MCA from a microcatheter (SL-10, Boston Scientific), and occlusion was documented using digital subtraction angiograms (DSAs) of the internal carotid and vertebrobasilar circulation every 15 min throughout the 1 h occlusion period. Following 1 h of MCAO, the embolic coil was retrieved and DSAs used to confirm reperfusion. Angiographic documentation of vessel perforation and hemorrhage was grounds for study exclusion. Physiological parameters were monitored throughout the procedure, and included blood pressure and blood parameters determined before MCAO, during occlusion, and after reperfusion. Following reperfusion, endovascular devices were withdrawn and arteriotomy sites closed. Under veterinary care, canines were immediately transported to the Wright Center of Innovation at The Ohio State University for 1 h post-reperfusion MRI. Fluoroscopy-guided angiograms documenting the surgical procedure are provided in FIGS. 8A-F.

### Example 3

### Magnetic Resonance Imaging (MRI)

Evaluation of the infarct volume was performed using an 8-channel sensitivity encoding (SENSE) knee coil in a 3T MRI (Achieva, Philips Healthcare) MRI imaging system. Images were obtained at 1 h and 24 h following reperfusion. Sequences included: diffusion tensor imaging (DTI) [field of view (FOV)=140×140 mm, matrix=128×128, number of excitations (NEX)=1, repetition time (TR)/echo time (TE) 192-2131/71, Slice thickness=3 mm, b value=1000, total scan time approximately 4 minutes] and T2 fluid attenuated inversion recovery (FLAIR) [FOV=160 mm, matrix=512×512, NEX=1, TR/TE/inversion time (TI)=11000/125/2800, slice thickness=3 mm, total scan time approximately 8 minutes] and 3D time-of-flight magnetic resonance angiography (MRA) (FOV=150 mm, matrix=512×512, TR/TE=8.6/3.45, flip angle=20, slice thickness=1 mm, total scan time approximately 6 minutes). DTI data were transferred to a workstation where mean diffusivity (MD) maps were derived from the one hour post reperfusion DTI (FSL 4.1.4, Oxford University). MRA reconfirmed reperfusion to the transiently occluded territory. Infarct volumes were calculated by importing MD maps and FLAIR images into Image J (National Institutes of Health). Two blinded observers independently outlined infarct volumes using a semi-automated threshold technique.

### Example 4

### Streamline and Probabilistic White Matter Fiber Tracking

Streamline tractography of the internal capsule was performed using the FACT algorithm with Trackvis software (ver. 0.5.1). Probabilistic tractography enables quantitative analysis of DTI based connectivity as opposed to the streamline tractography. To investigate the therapeutic efficacy of TE to protect white matter connectivity following stroke, a probabilistic tractography framework was employed using the FSL software package. The probabilistic approach used employed a single ROI mask with 10,000 tracts cast from each voxel in the internal capsule ROI (curvature threshold of 0.2). The connectivity images resided in their native space and were not directly comparable. For this reason, tensor images for each sample, for each timestamp, were fed into a tensor field based elastic registration routine to compute a population average tensor image and the transformations that mapped each data onto this average brain space. This registration was performed using DTI-TK toolkit (ver. 2.0). Transformations were applied to the corresponding tract images in the same coordinate framework, that of the mean tensor image.

### Example 5

### Angiographic Evaluation of Cerebrovascular Collateral Recruitment

DSA acquisitions obtained just prior to reperfusion were reviewed to assess cerebrovascular collateral recruitment using an 11-point scale. This scale takes into account the anatomic extent and transit time of leptomeningeal collaterals from the posterior (PCA) and anterior cerebral artery (ACA) circulations to the affected MCA territory. DSA images were reviewed to identify leptomeningeal collateral reconstitution of the anterior, middle and posterior aspects of the MCA territory. The horizontal portions of the MCA and PCA were used as landmarks dividing the MCA territory into these three regions—anterior, middle and posterior. Images were compared to the arterial and venous phases of the pre-occlusion arteriograms on the side of the occlusion

### Example 6

### Vitamin E Extraction and Analysis

Vitamin E extraction and analysis of canine brain tissue was performed using a HPLC-coulometric electrode array detector (Coularray Detector, 12-channel, model 5600, ESA Inc). This system enables the simultaneous detection of all eight naturally occurring vitamin E family members in a single run.

### Example 7

### Laser Microdissection Pressure Catapulting

Following 24 h MRI, canines were euthanized and brain tissue collected for downstream applications, including laser microdissection pressure catapulting (LMPC). Continuous coronal slices (3 mm) of canine brain which include the M1 segment of the MCA were embedded and frozen in OCT compound (Sakura). Embedded brains were sliced into 12 μm this sections using a cryostat (CM3050s, Leica Microsystems Inc). Sections were mounted onto RNAse inhibitor-treated thermoplastic (polyethylene napthalate)-covered glass slides (PALM Technologies). Slides were incubated in RNA-later stabilization reagent (Applied Biosystems) for 4 mins and quick-stained with anti-VWF antibody (1:50 dilution, 15 min) for selective capture of endothelial cells from stroke-affected (ipsilateral) and contralateral control tissue. More than 800,000 μm2 of capture elements were collected for downstream RNA isolation, cDNA synthesis and real-time PCR. For high-throughput collection, all elements were captured using a PALM MicroLaser, MicroBeam, and RoboStage/RoboMover system. RNA was isolated from captured and catapulted elements using the PicoPure RNA Isolation Kit (Arcturus).

### Example 8

### Real-Time PCR

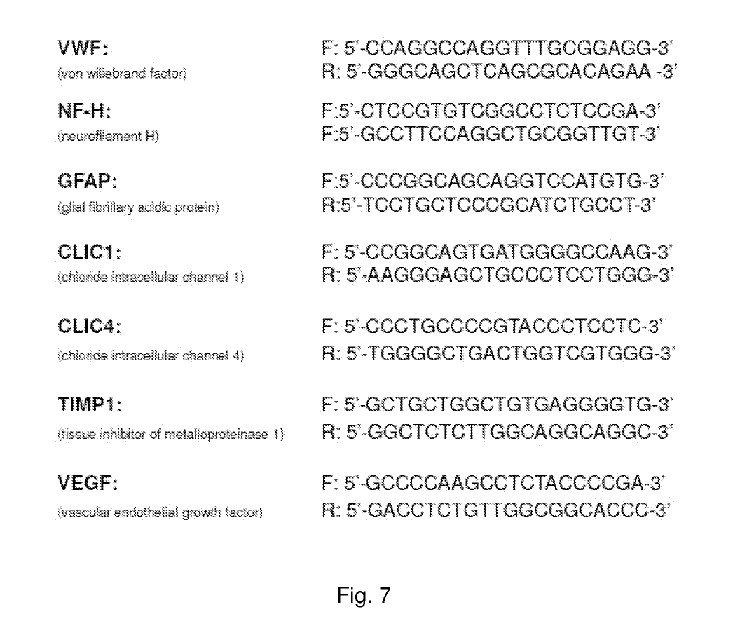

Expression levels of collateral gene candidates were independently determined at 24 h from contralateral control and stroke affected LMPC captured elements using real-time PCR. Briefly, total RNA (>250 ng) was reverse transcribed into cDNA using oligo-dT primer and Superscript III. RT-generated DNA was quantified by real-time PCR assay using double-stranded DNA binding dye SYBR Green-I. Relative gene expression was standardized to 18s rRNA. Data are shown as means±SD. Primer sequences are provided in FIG. 7.

### Example 9

### Western Blot Analysis

To extract protein from the canine brain, 51 cortex and contralateral control tissue was homogenized on ice in lysis buffer (50 mM Tris-HCL pH 7.6; 1.5 mM NaCl; 0.5 mM CaCl2; 0.01% Brij 35; 1% Triton X-100) and centrifuged at 4° C. for 15 minutes at 14,000 g. Protein expression of matrix metalloproteinase-2 (MMP2) in canine cortex was determined by Western blot analysis using MMP2 antibody (Enzo Life Sciences, PA). Proteins were separated on 4-12% gels (Invitrogen) by SDS-PAGE, transferred onto polyvinylidene difluoride (PVDF) membranes, and membranes were incubated with Tris-buffered saline (TBS) containing 5% milk for 12-18 h at 4° C. with MMP2 antibody (1:400 dilution). Next, membranes were washed three times with Tris-buffered saline containing 0.1% Tween-20 (TBST) and incubated for 1 h at room temperature in horseradish peroxidase-conjugated secondary donkey anti-rabbit antibody (GE Healthcare Life Sciences, NJ, 1:2000 dilution in TBST containing 5% milk) Immunoblots were developed with ECL Plus™ Western blotting Detection Reagents (GE Healthcare Life Sciences) according to manufacturer's recommendation. To evaluate the loading efficiency, the membranes were probed with anti-β-actin antibody (Sigma-Aldrich, 1:5000, in TBS, 1 h). Each Western blot was scanned and analyzed using National Institutes of Health ImageJ software (ver. 1.44) for the density of the bands.

### Example 10

### Gelatin Zymography

MMP2 activity was determined by gelatin zymography as described (Beceriklisoy et al 2007). Briefly, 50 μg total protein were combined in a 1:1 ratio with Tris Glycerine SDS loading buffer (Invitrogen, CA) and samples were separated through electrophoresis on 10% polyacrylamide gels containing 0.1% gelatin (Invitrogen). Gels were incubated in renaturing buffer (Invitrogen) for 30 mins, and then treated in developing buffer (Invitrogen) for 30 mins Gels were incubated for 24 h at 37° C. in fresh developing buffer with gentle agitation. Gels were stained with 20 ml of SimplyBlue™ SafeStain (Invitrogen), destained, and imaged using Pharos FX plus molecular imager (Bio-Rad, CA) and analyzed using National Institutes of Health ImageJ software (ver. 1.44) for the density of the bands.

### Example 11

### Statistical Analysis

Statistically treated data are reported as mean±standard deviation. Difference between means was tested with Student's t test or one-way ANOVA with Tukey's post-hoc test where appropriate (alpha level=0.05). SPSS software (v17.0) was used for all statistical calculations.

### Example 12

### Oral TE Supplementation Attenuates Stroke-Induced Lesion Volume and Edema

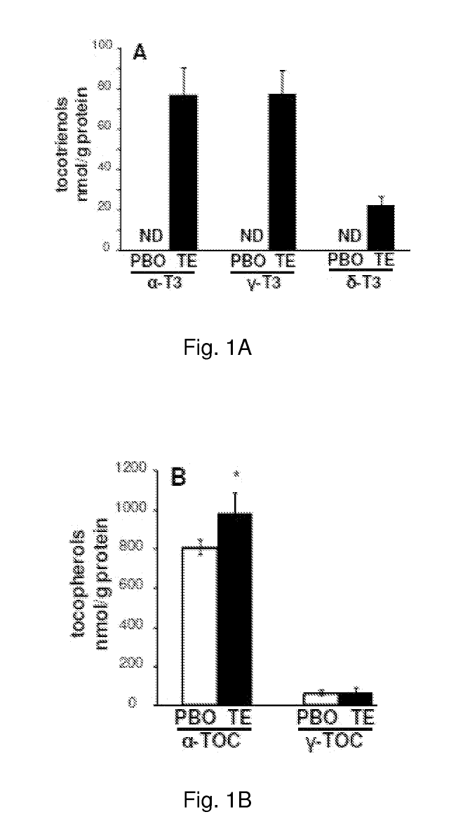

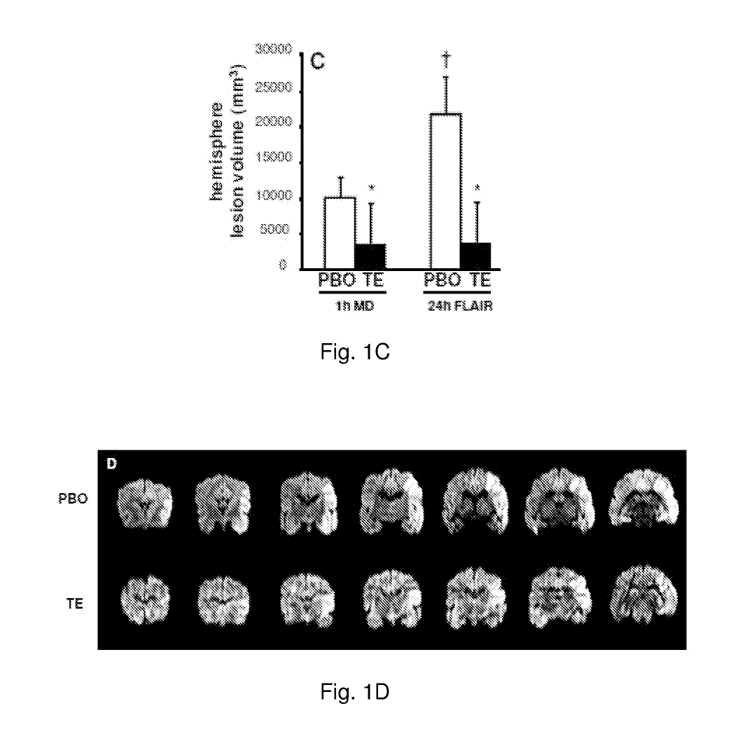

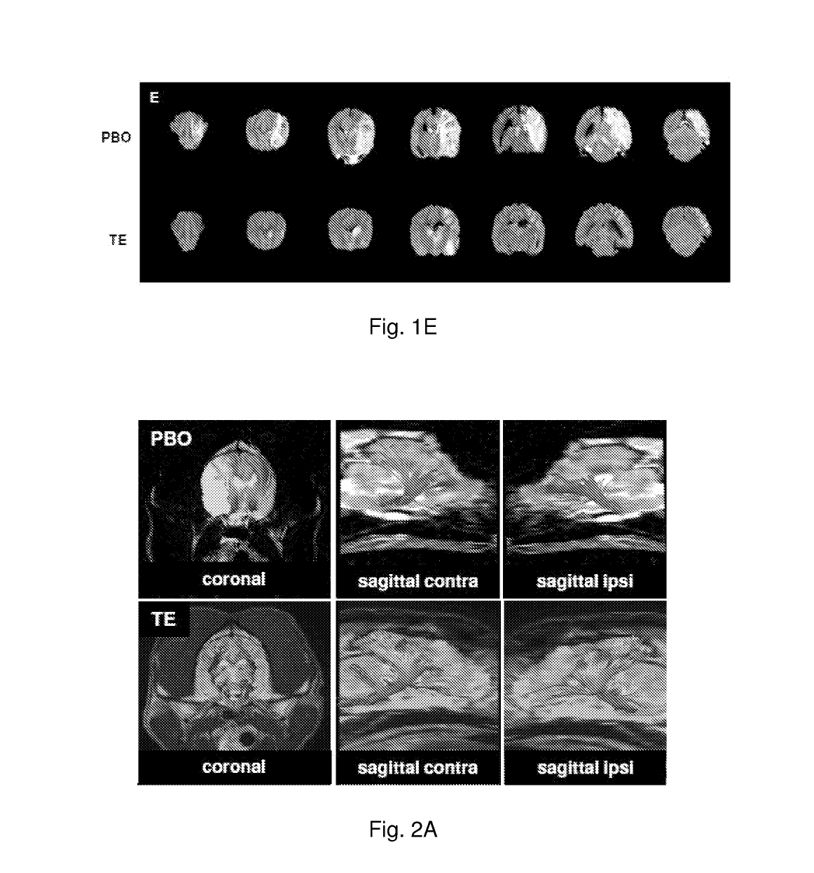

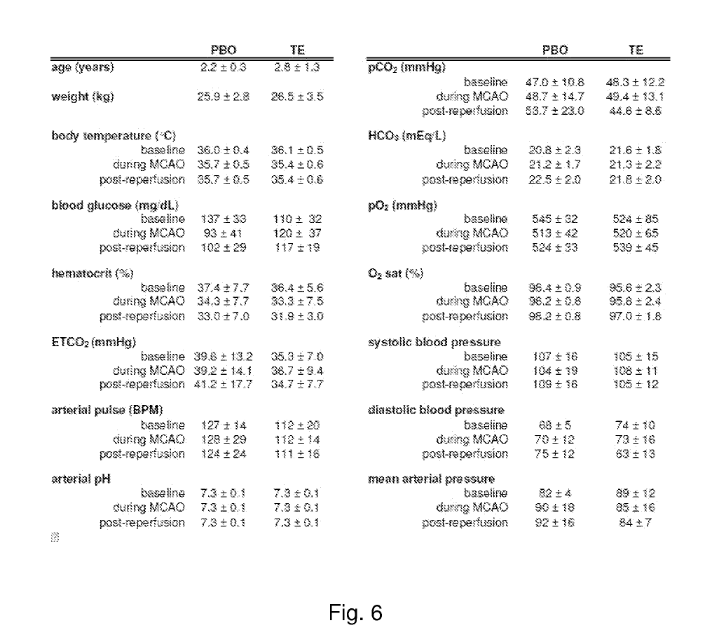

A. Healthy mongrel canines were randomized to treatment groups and orally administered 200 mg tocotrienol enriched (TE, containing 61.52 mg α-tocotrienol, 112.8 mg γ-tocotrienol, and 25.68 mg δ-tocotrienol; n=11) or vehicle control (PBO, placebo containing vitamin E stripped corn-oil, n=9) gel capsules bi-daily for ten weeks prior to experimental stroke. Randomization was supervised by the trial statistician, while research and veterinary personnel were blinded to supplement content and experimental groups until the conclusion of the study. TE supplementation had no significant effect on monitored physiological parameters prior to (baseline), during, or immediately following stroke reperfusion (FIG. 6). Oral TE capsule supplementation significantly increased the concentration of tocotrienols in middle cerebral artery (MCA) supplied cerebral cortex as compared to PBO controls (FIG. 1A). TE supplementation enriched cortical brain tissue with nearly equal amounts of α- and γ-TCT isoforms (77.4 nmol/g protein and 77.5 nmol/g protein respectively) and approximately one-third that amount of δ-tocotrienol isoform (22.4 nmol/g protein). Like Western diet, canine chow is deficient in tocotrienols. No appreciable amount of α-, γ-, or δ-tocotrienol was detected in cortex of PBO controls despite using a highly-sensitive electrochemical HPLC approach (Roy et al 2002). The concentration of α- and γ-tocotrienol in TE supplemented animals was 10-fold less than that of α-tocopherol found in cerebral cortex (FIG. 1B). TE supplementation, representing a blend of natural vitamin E enriched from palm oil, modestly increased the concentration α-tocopherol in brain tissue as compared to PBO controls; while no difference in γ-tocopherol concentration was observed between PBO and TE groups.

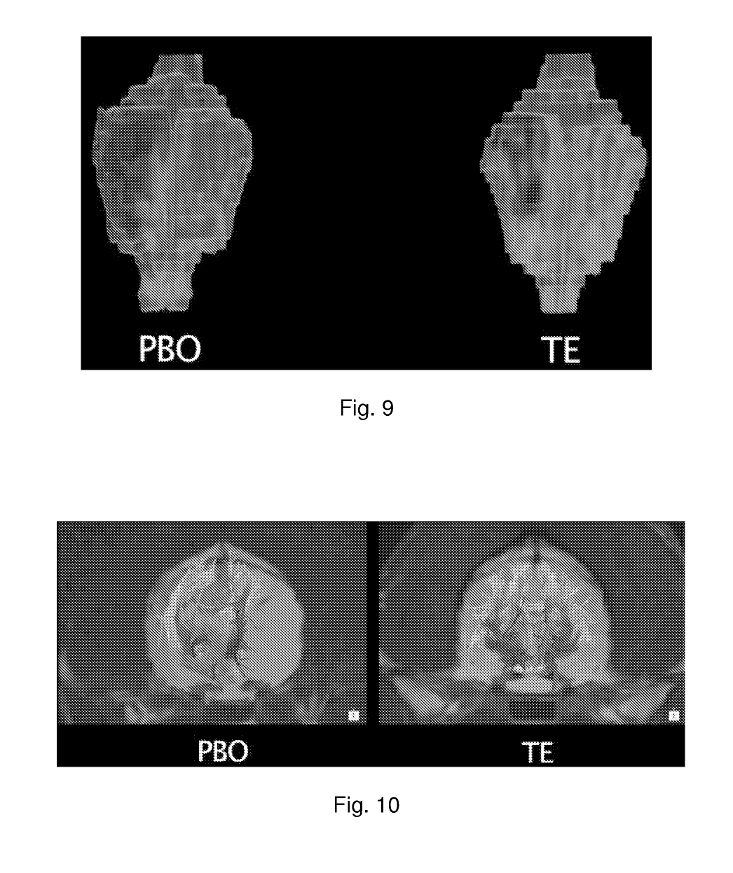

B. Cytotoxic edema is characterized by cellular swelling in the acute phase (<24 h) of stroke onset. Cerebral ischemia in hyper-metabolic brain tissue causes failure of ATP-dependent ion transporters, resulting in rapid accumulation of intracellular Na2+ and an influx of water to maintain osmotic equilibrium. DTI enables early detection of cytotoxic edema following acute ischemic stroke. Mean diffusivity maps generated from DTI revealed that TE supplemented canines had significantly attenuated (p<0.05) cytotoxic edema at 1 h following acute ischemic stroke as compared to PBO controls (FIG. 1C, 1D). While stroke-induced lesion volume more than doubled in PBO canines between the 1 h and 24 h (9804.7 mm3 to 20579.8 mm3) time-points after reperfusion, lesion volume in TE supplemented canines remained consistently low (3675.3 mm3 to 3834.9 mm3, FIG. 1C, 1E). At the 1 h time-point, stroke-induced lesion volume of TE supplemented canines was <40% that of PBO controls; and at 24 h TE infarct volume was <20% of their PBO counterparts. Three-dimensional volumetric reconstruction of brain from representative PBO and TE FLAIR images at 24 h provides a clear visual appreciation of the protective effects of TE supplementation (FIG. 9).

### Example 13

### White Matter Fiber Tract Connectivity is Protected in TE Supplemented Canines Following Stroke

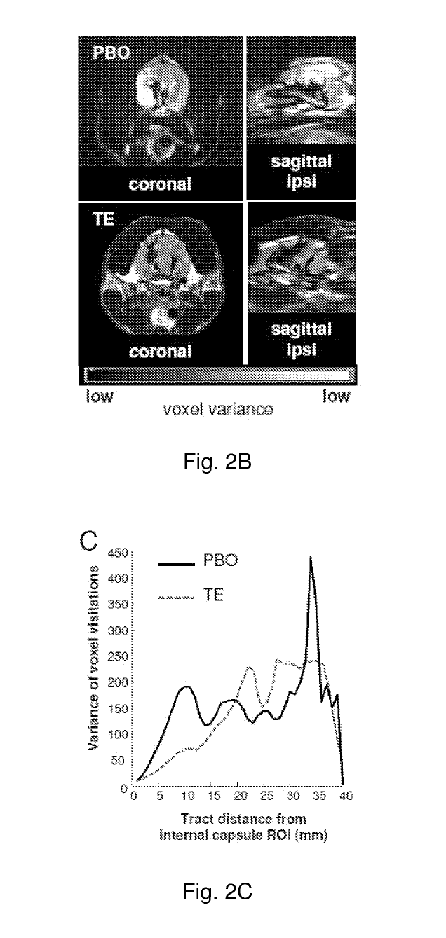

White matter fiber pathways represent the brain's communication network. The cytoarchitecture and anatomical connectivity of cerebral white matter with cerebral cortex (gray matter) directly influences brain function. White matter injury in the context of stroke has a direct effect on sensorimotor impairment and post-stroke functional recovery. In brain tissue that possesses a high degree of directional organization, the diffusion of water and its protons aligns with the orientation of white matter fiber tracts. Recent developments in DTI have enabled visualization of white matter fiber tract connectivity following stroke. Fiber tract projections from the region of the internal capsule to the corona radiata were dramatically reorganized in PBO canine brain 24 h after stroke reperfusion (FIGS. 2A, 10). Specifically, streamline tractography visualization of fiber tracts revealed impaired connectivity between regions of interest (ROI) set in the internal capsule and corona radiata. Oral TE supplementation protected fiber tract projections in the stroke-affected hemisphere as compared to PBO control. Probabilistic tractography is a powerful tool for quantitative analysis of white matter connectivity. The inventors employed a probabilistic tractography framework to quantitatively assess the effect of TE supplementation on white matter fiber tract connectivity in stroke-affected cortex. To quantitatively assess connectivity, 40,000 tracts were cast from voxels in the internal capsule ROI to the distal corona radiata ROI (FIG. 2B). Relative connectivity of fiber tracts between the internal capsule and corona radiata was much higher in representative TE supplemented canine brain as compared to PBO control. The PBO canine brain had a higher tract variance as a function of distance from the internal capsule seed ROI as compared to the TE counterpart (FIG. 2C).

### Example 14

### TE Supplementation Improved Cerebrovascular Collateral Circulation During Ischemic Stroke

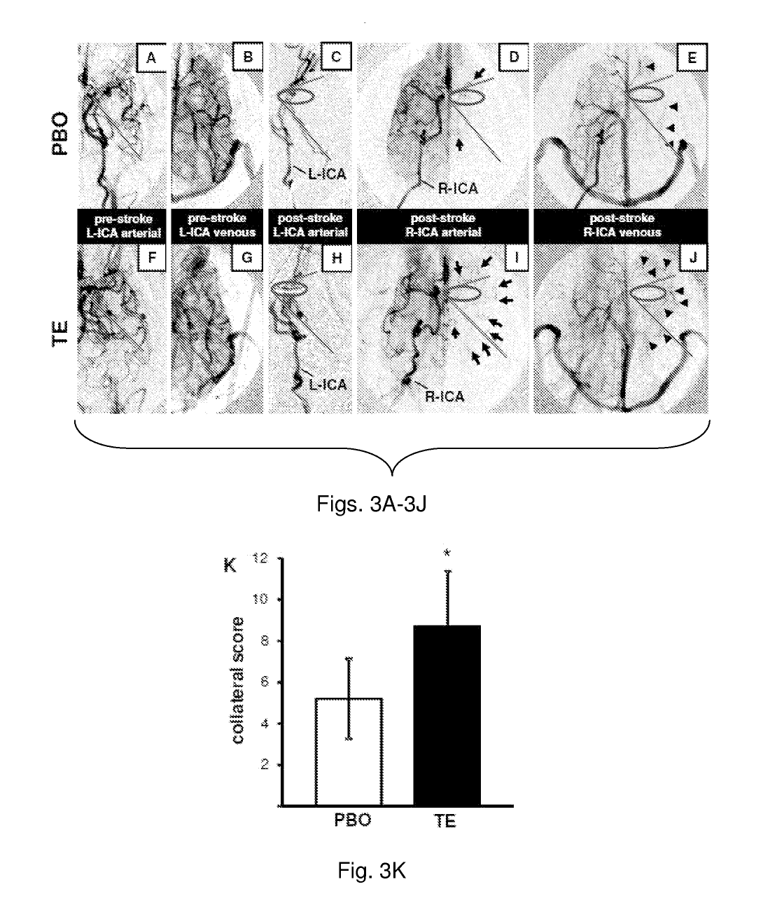

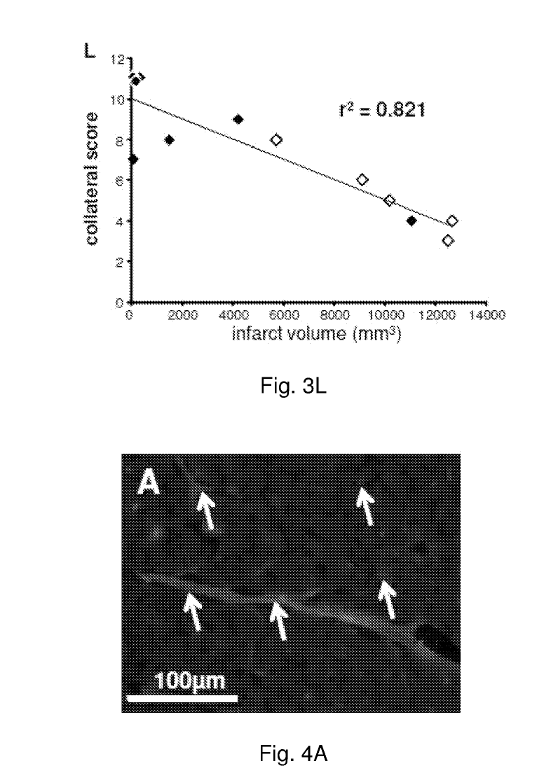

Collateral arteries of the leptomeningeal space anastomose across border zones of cortical watersheds in humans and large mammals alike underscoring the translational significance of the inventors' approach. This arterial network facilitates an alternative means to circulate blood, via retrograde filling, to tissue in instances when injury or occlusion to primary cortical branches disrupts cerebrovascular blood flow. Improving collateral circulation and blood perfusion to the stroke affected territory is a therapeutic target of recognized value in the clinic. In many cases, a focal circulatory abnormality created by arterial occlusion can be adequately compensated through cerebrovascular collateral circulation. The inventors' pre-clinical canine stroke model benefits from angiographic assessment of collateral circulation during MCAO. Post-hoc analysis of cerebral angiograms during ischemic stroke revealed that canines receiving oral TE supplementation had improved cerebrovascular collateral circulation as compared to PBO controls FIG. 3. Pre- and post-MCAO internal carotid artery angiograms (FIG. 3A-J) enable objective scoring of stroke-affected hemisphere collaterals according to a clinically relevant 11-point scale. MCA-territory collateral score was significantly higher in TE supplemented canines as compared to PBO controls (PBO=5.2±1.9, TE=8.1±2.9; FIG. 3K). A higher collateral score, and therefore better perfusion in the stroke-affected hemisphere, tightly correlated with smaller stroke-induced lesion size at 24 h (r2=0.821, FIG. 3L).

### Example 15

### TE Induced Expression of Arteriogenic Genes in Cerebral Cortex Collaterals

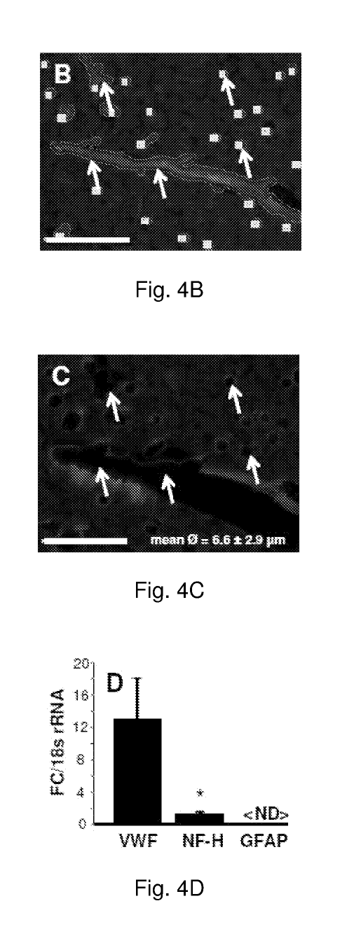

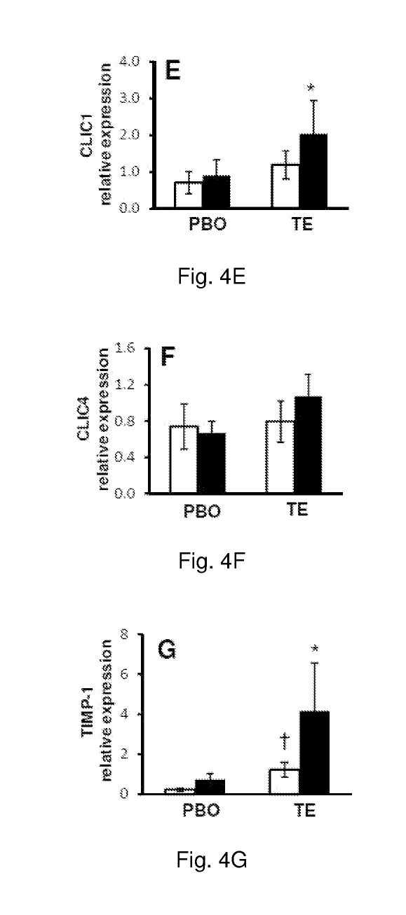

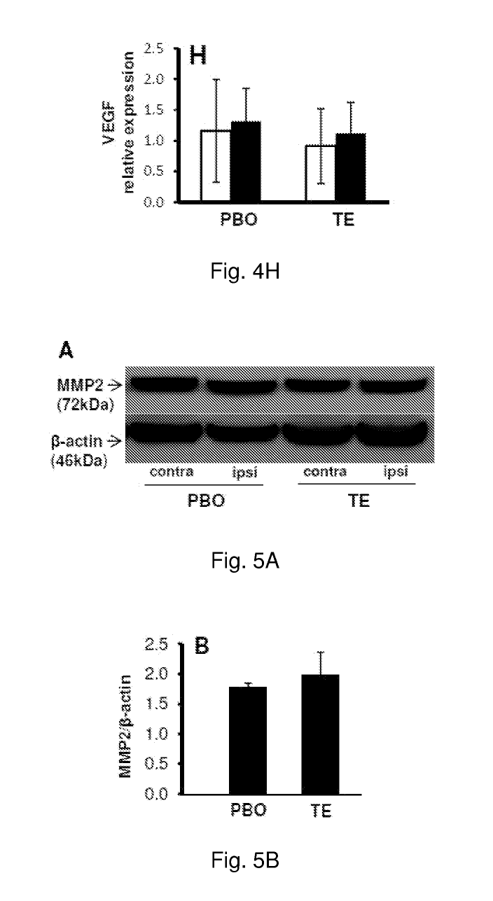

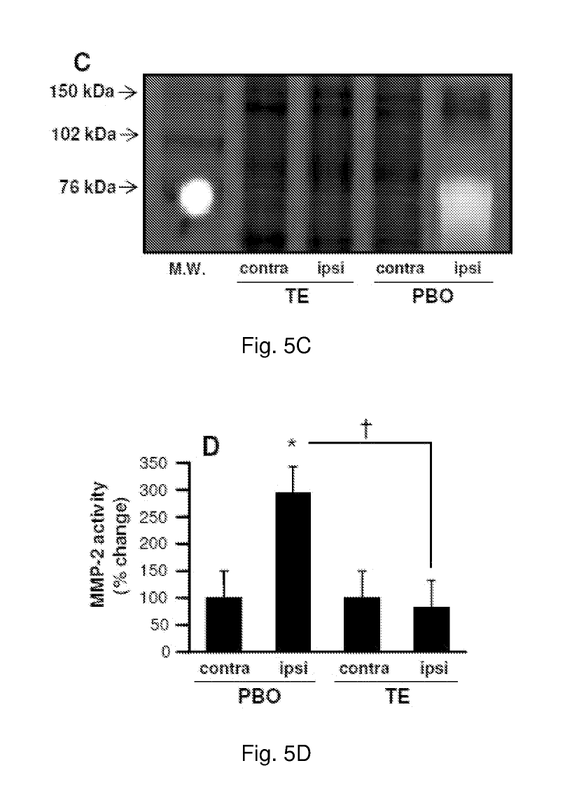

Arteriogenesis refers to a positive outward remodeling of pre-existing collateral arteries into larger vessels, which bypass sites of occlusion. To determine whether TE supplementation invoked molecular mechanisms of cerebral arteriogenesis, arterioles from the stroke-affected (ipsilateral) and contralateral control cerebral cortex were selectively isolated LMPC (FIG. 4A-D). Known gene targets of cerebral arteriogenesis include members of the chloride intracellular channel (CLIC), tissue inhibitor of metalloprotease 1 (TIMP1), and vascular endothelial growth factor (VEGF). Increased gene expression of CLIC1 and TIMP1 was observed in stroke affected cortex of TE supplemented canines as compared to PBO controls (FIG. 4E, G). Of particular note, TE supplementation dependent increase in TIMP1 expression was not limited to stroke-affected endothelial cells at the ipsilateral site. TE supplementation induced TIMP1 in arterioles captured from contralateral control tissue (FIG. 4G). These effects were specific as other arteriogenic candidate genes such as CLIC4 and VEGF were not affected by TE supplementation (FIG. 4F, H). TIMP1 binds to active matrix metalloprotease-2 (MMP2) in a 1:1 stoichiometric ratio, providing localized control of MMP activity. Independent of MMP2 protein expression (FIG. 5A, B), TE supplementation significantly attenuated MMP2 activity in the stroke-affected cerebral cortex (FIG. 5C, D).

While a number of exemplary aspects and embodiments have been discussed above, those of skill in the art will recognize certain modifications, permutations, additions and sub-combinations therefore. It is therefore intended that the following appended claims hereinafter introduced are interpreted to include all such modifications, permutations, additions and sub-combinations are within their true spirit and scope. Each apparatus embodiment described herein has numerous equivalents.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims. Whenever a range is given in the specification, all intermediate ranges and subranges, as well as all individual values included in the ranges given are intended to be included in the disclosure. When a Markush group or other grouping is used herein, all individual members of the group and all combinations and subcombinations possible of the group are intended to be individually included in the disclosure.

In general the terms and phrases used herein have their art-recognized meaning, which can be found by reference to standard texts, journal references and contexts known to those skilled in the art. The above definitions are provided to clarify their specific use in the context of the invention.

What is claimed is:

1. A method of improving pial collateral circulation and protecting ischemic tissue, comprising:
a.) administering a composition comprising at least one form of tocotrienol in an amount of from about 10 mg to about 1000 mg per day to a subject in need of pial collateral circulation improvement and ischemic tissue protection; and
b.) improving pial collateral circulation and protecting ischemic tissue in the subject.

2. A method of promoting arteriogenesis in a subject comprising:
a.) administering a composition comprising at least one form of tocotrienol in an amount of from about 10 mg to about 1000 mg per day to a subject in need of arteriogenesis promotion; and
b.) promoting arteriogenesis in the subject.

3. A method of increasing Tissue Inhibitor of Metalloproteinase Metallopeptidase Inhibitor 1 (TIMP1) in vessels of cerebrovascular collateral circulation and attenuating the activity of Matrix Metalloproteinase-2 (MMP2) in a subject in need thereof, comprising:
a.) administering a composition comprising at least one form of tocotrienol in an amount of from about 10 mg to about 1000 mg per day to a subject in need of an increase in Tissue Inhibitor of Metalloproteinase Metallopeptidase Inhibitor 1 (TIMP1) in vessels of cerebrovascular collateral circulation and in need of attenuation of the activity of Matrix Metalloproteinase-2 (MMP2); and
b.) increasing Tissue Inhibitor of Metalloproteinase Metallopeptidase Inhibitor 1 (TIMP1) in vessels of cerebrovascular collateral circulation and attenuating the activity of Matrix Metalloproteinase-2 (MMP2) in the subject.

4. A method of ameliorating the symptoms of cerebral blood clotting, or reducing the risk of cerebral blood clotting, in a subject comprising:
a.) administering a composition comprising at least one form of tocotrienol in an amount of from about 10 mg to about 1000 mg per day to a subject in need of amelioration of the symptoms of cerebral blood clotting, or reducing of risk of cerebral blood clotting; and
b.) ameliorating the symptoms of cerebral clotting, or reducing risk of cerebral blood clotting in the subject.

5. A method of claim 4, wherein the cerebral blood clotting is associated with a cerebrovascular ischemic disease, wherein the disease is selected from the group consisting of:
a.) cerebral ischemia, in particular transient ischemic attack, stroke, vascular dementia and/or infarct dementia;
b.) myocardial ischemia, in particular a coronary heart disease and/or myocardial infarction; and/or
c.) peripheral limb disease, in particular periphery arterial occlusive disease.

6. A method of claim 1, which further has an effect on the subject selected from the group consisting of: attenuating ischemic stroke-induced lesion volume; preventing loss of white matter fiber tract connectivity following stroke; improving cerebrovascular collateral circulation; preventing blood vessel injury; reducing the risk of ischemic stroke; reducing cerebrovascular ischemic disease; and ameliorating the symptoms of obstruction of a blood vessel.

7. A method of claim 1, wherein 100 mg and 500 mg per day of at least one tocotrienol is administered via oral supplementation, for at least four weeks.

8. A method of claim 1, wherein the at least one tocotrienol is selected from the group consisting of: α tocotrienol; β tocotrienol; γ tocotrienol; δ tocotrienol; and combinations thereof.

9. A method of claim 1, wherein the composition comprises mixed tocotrienols enriched to a percentage of the total weight of the composition selected from the group consisting of: approximately 20%; approximately 30%; approximately 40%; approximately 50%; approximately 60%; approximately 70%; approximately 80%; and approximately 90%.

10. A method of claim 1, wherein the at least one tocotrienol is administered as 400 mg daily dose of Tocovid Suprabio®, for at least four weeks.

11. A method of claim 1, wherein the oral supplement is delivered by one or more of: a capsule; a tablet pill; a colloid; a piece of chewing gum; a gel; a drink; a food additive; a thin film dissolving strip; an emulsified food spread; an emulsion, a syrup; a meat food; a dairy food; and an egg.

12. A method of claim 4, wherein the subject is at elevated risk for cerebral blood clotting.

13. A method of claim 1, which further comprises administering a blood-thinning agent.

14. A method of claim 1, wherein the subject is selected from the group consisting of: human; livestock; companion animal; research animal.

15. A method of claim 1, wherein the subject is selected from the group consisting of: astronaut; pilot; professional racecar driver; deep-sea diver; mountain climber; pre-surgery patient; sickle-cell anemia patient; sleep apnea patient; drug rehabilitation patient; elderly person; elderly animal; greyhound; or racehorse.

16. A method of claim 1, wherein said subject has an attribute selected from the group consisting of:
a.) showing symptoms of being at risk of developing the cerebrovascular ischemic disease;
b.) showing any risk markers in ex vivo tests, in particular in blood samples;
c.) has previously had a cerebral or myocardial ischemia; and/or
d.) has a predisposition of developing a cerebrovascular ischemic disease, in particular a genetic predisposition.

17. A method of claim 1, wherein the symptoms are selected from the group comprising: neurological malfunctions, transitory ischemic attack, congestive heart failure, angina pectoris, valvular heart disease, cardiomyopathy, pericardial disease, congenital heart disease, coarctation, atrial and/or ventricular septal defects.

18. A method of claim 1, wherein the subject exhibits at least one condition selected from the group consisting of: Alzheimer's disease; sclerosis, in particular atherosclerosis and/or transplantation-induced sclerosis; a cerebral occlusive disease, renal occlusive disease, a mesenterial artery insufficiency or an ophthalmic or retinal occlusion, post-operative or post-traumatic condition; thrombosis; embolism; restenosis, in particular primary restenosis, secondary restenosis and/or in-stent restenosis; trisomy 21; hypoglycemia; vasculitis; preeclampsia; placental hypoxia; sleep apnea; sexual dysfunction, in particular erectile dysfunction or female sexual dysfunction; post-operative hypoxia; Raynaud's disease; endothelial dysfunction; cancer; renal failure; varicose veins; edema; hypotension; decubitus; carbon monoxide poisoning; heavy metal poisoning; ulcers; sudden infant death syndrome; erythroblastosis; asthma; chronic obstructive pulmonary disease; sickle cell disease; induced g-forces which restrict the blood flow and force the blood to the extremities of the body; caisson's disease; localized extreme cold, in particular frost bite; tourniquet application; an increased level of glutamate receptor stimulation or for any disease where atherosclerosic plaques in the vascular wall lead to an obstruction of the vessel diameter; osteonecrosis; and Legg Calvé Perthes disease.

19. A method of claim 1, wherein said subject has been, or will be, exposed to:
a.) a pharmaceutical or medical treatment damaging one or more arteries;
b.) a radiation treatment damaging one or more arteries; and/or
c.) a surgical treatment damaging one or more arteries.

20. A pharmaceutical composition for the treatment of an obstruction in a blood vessel comprising: one or more thrombolytic drugs, and one or more tocotrienol-comprising composition.

21. A pharmaceutical composition herein, wherein the tocotrienol-comprising composition is Tocovid Suprabio®.

22. A pharmaceutical composition herein, which further comprises one or more compounds selected from the group consisting of: heparin; tocopherols; one or more colony stimulating factor(s) (CSF(s)); one or more icosanoid(s); angiotensin converting enzyme (ACE) inhibitors; beta-blockers; antiplatelet agents; pentoxifylline; and cilostazol.

23. A nutritional supplement to aid circulatory health comprising, at least one tocotrienol and at least two additional compounds selected from: vitamin A, vitamin B, vitamin C, vitamin D, grape seed extract, hawthorn extract, green tea extract, garlic extract, limonene, carnitine, lutein, zeaxanthin, omega-3 essential fatty acids, zinc, calcium, chromium, and iron.

24. A nutritional supplement of claim 23, wherein the additional compounds selected are fat-soluble.

24. An infant formula composition comprising fat, carbohydrate, protein, and vitamins wherein at least one of the vitamins is a tocotrienol.


SEQUENCE LISTING (14 sequences)

SEQ ID NO: 1 - DNA, 20 bp, Artificial Sequence
  Description of Artificial Sequence Synthetic primer
ccaggccaggtttgcggagg

SEQ ID NO: 2 - DNA, 20 bp, Artificial Sequence
  Description of Artificial Sequence Synthetic primer
gggcagctcagcgcacagaa

SEQ ID NO: 3 - DNA, 20 bp, Artificial Sequence
  Description of Artificial Sequence Synthetic primer
ctccgtgtcggcctctccga

SEQ ID NO: 4 - DNA, 20 bp, Artificial Sequence
  Description of Artificial Sequence Synthetic primer
gccttccaggctgcggttgt

SEQ ID NO: 5 - DNA, 20 bp, Artificial Sequence
  Description of Artificial Sequence Synthetic primer
cccggcagcaggtccatgtg

SEQ ID NO: 6 - DNA, 20 bp, Artificial Sequence
  Description of Artificial Sequence Synthetic primer
tcctgctcccgcatctgcct

SEQ ID NO: 7 - DNA, 20 bp, Artificial Sequence
  Description of Artificial Sequence Synthetic primer
ccggcagtgatggggccaag

SEQ ID NO: 8 - DNA, 20 bp, Artificial Sequence
  Description of Artificial Sequence Synthetic primer
aagggagctgccctcctggg

SEQ ID NO: 9 - DNA, 20 bp, Artificial Sequence
  Description of Artificial Sequence Synthetic primer
ccctgccccgtaccctcctc

SEQ ID NO: 10 - DNA, 20 bp, Artificial Sequence
  Description of Artificial Sequence Synthetic primer
tggggctgactggtcgtggg

SEQ ID NO: 11 - DNA, 20 bp, Artificial Sequence
  Description of Artificial Sequence Synthetic primer
gctgctggctgtgaggggtg

SEQ ID NO: 12 - DNA, 20 bp, Artificial Sequence
  Description of Artificial Sequence Synthetic primer
ggctctcttggcaggcaggc

SEQ ID NO: 13 - DNA, 20 bp, Artificial Sequence
  Description of Artificial Sequence Synthetic primer
gccccaagcctctaccccga

SEQ ID NO: 14 - DNA, 20 bp, Artificial Sequence
  Description of Artificial Sequence Synthetic primer
gacctctgttggcggcaccc